(19) United States
(12) Patent Application Publication
(10) Pub. No.: US 2024/0209416 A1
(43) Pub. Date: Jun. 27, 2024

(54) METHOD FOR MODIFYING SURFACE OF SUBSTRATE AND METHOD FOR IMMOBILIZING OLIGONUCLEOTIDE

(71) Applicant: Industrial Technology Research Institute, Hsinchu (TW)

(72) Inventors: Wei-Chin Huang, Tainan City (TW); An-Li Chen, Tainan City (TW); Ming-Chun Lin, Hsinchu City (TW); Chun-Ting Lai, Taoyuan City (TW); Li-Cheng Jheng, Tainan City (TW); Sung-Ho Liu, Kaohsiung City (TW); Bo-Huai Wang, Kaohsiung City (TW)

(73) Assignee: Industrial Technology Research Institute, Hsinchu (TW)

(21) Appl. No.: 18/191,864

(22) Filed: Mar. 28, 2023

(30) Foreign Application Priority Data
Dec. 23, 2022 (TW) 111149811

(51) Int. Cl. C12Q 1/6811; C08L 33/26

(52) U.S. Cl. CPC C12Q 1/6811; C08L 33/26

(57) ABSTRACT

A method for modifying a surface of a substrate and a method for immobilizing oligonucleotide are provided. The method includes the following steps. A substrate is provided. A branched polymer is grafted on a surface of the substrate, wherein a side chain of a repeating unit of the branched polymer has a group capable of binding to an oligonucleotide. One end of the branched polymer has a group capable of binding to the surface of the substrate.

### CROSS-REFERENCE TO RELATED APPLICATION

This application claims the priority benefit of Taiwan application serial no. 111149811, filed on Dec. 23, 2022. The entirety of the above-mentioned patent application is hereby incorporated by reference herein and made a part of this specification.

### TECHNICAL FIELD

The disclosure relates to a method for modifying a surface of a substrate and a method for immobilizing oligonucleotide.

### BACKGROUND

Significant progress has been made in recent years in the sequencing and decoding of whole genome, especially human whole genome, thus driving the need for advances in nucleic acid synthesis technology. Currently, DNA sequencing is performed by nucleic acid solid-phase synthesis method. In this method, it is usually necessary to immobilize the oligonucleotide as the linker on the reactor. At present, the common solid-phase synthesis method is granule-based, which mainly grafts linkers for nucleic acid synthesis to polymer particles or glass beads through a single short-chain or long-chain. However, the above-mentioned method has the problems of poor grafting efficiency and high cost due to the single chain.

### SUMMARY

According to an embodiment of the disclosure, a method for modifying a surface of a substrate includes the following steps. First, a substrate is provided. Next, a branched polymer is grated on a surface of the substrate, wherein a side chain of a repeating unit of the branched polymer includes a group capable of binding to an oligonucleotide, and one end of the branched polymer includes a group capable of binding to the surface of the substrate.

According to an embodiment of the disclosure, a method for modifying a surface of a substrate includes the following steps. First, a substrate is provided. Next, a branched polymer including a first repeating unit and a second repeating unit is grafted on a surface of the substrate, wherein a side chain of the first repeating unit of the branched polymer has a group capable of binding to the surface of the substrate, and a side chain of the second repeating unit of the branched polymer has a group capable of binding to an oligonucleotide.

According to an embodiment of the disclosure, a method for immobilizing oligonucleotide includes the following steps. First, a substrate modified by using the aforementioned method for modifying the surface of the substrate is provided. Next, oligonucleotides are brought into to contact with the surface of the substrate.

Several exemplary embodiments accompanied with figures are described in detail below to further describe the disclosure in details.

### BRIEF DESCRIPTION OF THE DRAWINGS

The accompanying drawings are included to provide a further understanding, and are incorporated in and constitute a part of this specification. The drawings illustrate exemplary embodiments and, together with the description, serve to explain the principles of the disclosure.

FIG. 1 is a flow chart of modifying a surface of a substrate according to a first embodiment of the disclosure.

FIG. 2 is a flow chart of modifying a surface of a substrate according to a second embodiment of the disclosure.

FIG. 3 is a flow chart of modifying a surface of a substrate according to a third embodiment of the disclosure.

### DESCRIPTION OF THE EMBODIMENTS

As used herein, terms “about,” “approximately,” “essentially,” and “essentially” include the stated value and averages within acceptable deviations from the particular value as determined by one of ordinary skill in the art, taking into account the measurement in question and the specific amount of error associated with the measurement (i.e., the limitations of the measurement system). For example, the term “about” can mean within one or more standard deviations of the stated value, or for example within +30%, +20%, +15%, +10%, +5%. Furthermore, the terms “about”, “approximately”, “essentially” and “substantially” used herein can choose a more acceptable range of deviation or standard deviation according to the nature of measurement or other properties, instead of using a standard deviation apply to all properties.

In this specification, when no specific definition is provided otherwise, “(meth)acrylate” refers to both “acrylate” and “methacrylate”, and “(meth)acrylic acid” refers to “acrylic acid” and “methacrylic acid”.

In this specification, when no specific definition is otherwise provided, the term “combination” means mixture or copolymerization.

In this specification, when a definition is not provided otherwise, if a chemical bond is not drawn at a position that should be given in a chemical formula, hydrogen is bonded at the position.

In addition, in this specification, when no definition is provided otherwise, “*” means a connection point with the same or different atom or chemical formula.

The disclosure will be further described through the implementations below, but these implementations are only for illustrative purposes and not intended to limit the scope of the invention.

An embodiment provides a method for modifying a surface of a substrate, which can graft a branched polymer with a specific functional group to the surface of the substrate.

In an embodiment, a method for modifying a surface of a substrate includes the following steps. First, a substrate is provided. Next, a branched polymer is grated on a surface of the substrate, wherein a side chain of a repeating unit of the branched polymer includes a group capable of binding to an oligonucleotide, and one end of the branched polymer includes a group capable of binding to the surface of the substrate.

In an embodiment, a method for modifying a surface of a substrate includes the following steps. First, a substrate is provided. Next, a branched polymer including a first repeating unit and a second repeating unit is grafted on a surface of the substrate, wherein a side chain of the first repeating unit of the branched polymer has a group capable of binding to the surface of the substrate, and a side chain of the second repeating unit of the branched polymer has a group capable of binding to an oligonucleotide.

An embodiment provides a method for immobilizing oligonucleotide, which has high immobilization efficiency of oligonucleotide.

In an embodiment, a method for immobilizing oligonucleotide includes the following steps. First, a substrate modified by using the aforementioned method for modifying the surface of the substrate is provided. Next, oligonucleotides are brought into to contact with the surface of the substrate.

[The Method for Modifying the Surface of the Substrate of the Disclosure]

### First Embodiment

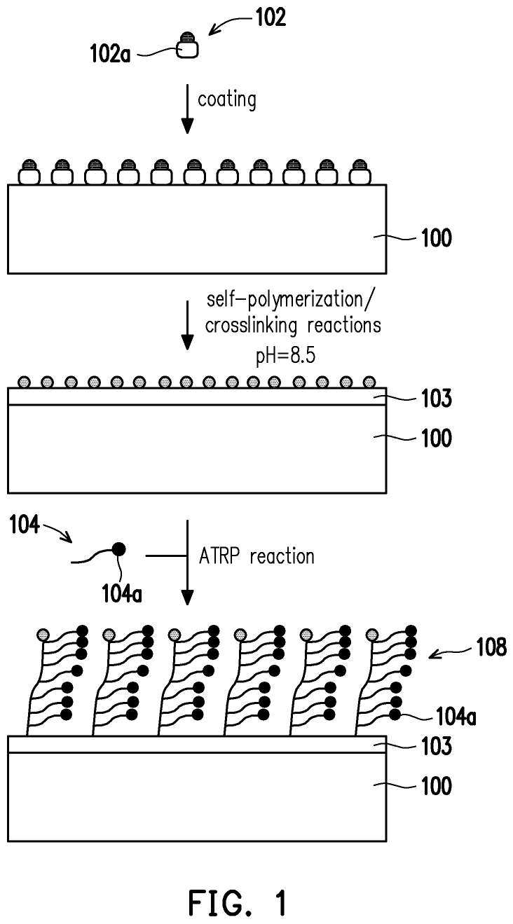

FIG. 1 is a flow chart of modifying a surface of a substrate according to a first embodiment of the disclosure.

The method for modifying the surface of the substrate according to the first embodiment of the disclosure includes the following steps. First, a substrate 100 is provided. In this embodiment, the material of the substrate 100 of the disclosure can include an inorganic material, an organic material or a metal. In one embodiment, the inorganic material can include silicon dioxide, graphite, ceramics or metal oxides, but the invention is not limited thereto. The organic material can include high molecular polymers or biomacromolecules, but the invention is not limited thereto. In an embodiment, the metal can include an alloy material. In an embodiment, the alloy material can include titanium alloy, aluminum alloy or stainless steel, but the invention is not limited thereto. In one embodiment, the substrate 100 of the disclosure is manufactured through 3D printing or general processing. In one embodiment, the substrate 100 of the disclosure is, for example, a microfluidic device, and a surface of the substrate 100 is, for example, an inner surface of the microfluidic device.

Next, a first modification composition including a polymerization initiator 102 is coated on the surface of the substrate 100. In this embodiment, one end of the polymerization initiator 102 of the disclosure has a group 102a capable of binding to the surface of the substrate. Specifically, the polymerization initiator 102 of the disclosure binds to the surface of the substrate 100 through the group 102a at the end thereof and capable of binding to the surface of the substrate. In one embodiment, the group 102a of the polymerization initiator 102 that can bind to the surface of the substrate is, for example, —OH group. In one embodiment, the group 102a of the polymerization initiator 102 that can bind to the surface of the substrate is, for example, a group having a dopamine structure, and the group having a dopamine structure can be fixed on the surface of the substrate through a phenolic hydroxyl group.

In this embodiment, the polymerization initiator 102 of the disclosure can react with the subsequent polymerizable monomer 104 to carry out a polymerization reaction. In one embodiment, the polymerization initiator 102 is, for example, a halide-based compound or an acyl halide-based compound. In one embodiment, the polymerization initiator 102 is, for example, a halide-based compound or an acyl-halide-based compound having a group at one end thereof and capable of binding to the surface of the substrate. In one embodiment, the polymerization initiator 102 is, for example, 2-bromoisobutyryl catechol derivatives. In one embodiment, the polymerization initiator 102 is, for example, 2-bromoisobutyryl dopamine (DA-BiBB) compound, which has the following structure.

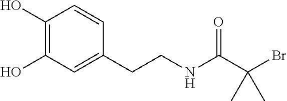 [SMILES: CC(C)(Br)C(=O)NCCc1ccc(O)c(O)c1]

In one embodiment, when the group 102a of the polymerization initiator 102 that can bind to the surface of the substrate is a group having a dopamine structure (such as a DA-BiBB compound), after the first modification composition including the polymerization initiator 102 is coated on the surface of the substrate 100, the first modification composition undergoes a self-crosslinking reaction in an alkaline environment. For example, the pH value of the first modification composition can be adjusted to an alkaline environment by using a common buffer. In the alkaline environment, the catechol of dopamine in the polymerization initiator 102 will be oxidized to o-quinones, which will undergo self-polymerization/crosslinking reactions to form polydopamine 103 on the surface of the substrate. The polydopamine 103 further improves the binding force of the polymerization initiator 102 and the substrate 100. In one embodiment, the alkaline environment is, for example, an environment with a pH value above 8.

Then, a second modification composition including a polymerizable monomer 104 is added to the first modification composition on the surface of substrate 100, to carry out a polymerization reaction and form a branched polymer 108.

In this embodiment, the polymerizable monomer 104 of the disclosure has a group 104a capable of binding to an oligonucleotide. In one embodiment, the group 104ₐ capable of binding to the oligonucleotide can include —NH₂ group or —OH group.

In one embodiment, the polymerizable monomer 104 can include a (meth)acrylate-based polymerizable monomer or a (meth)acrylamide-based polymerizable monomer. In one embodiment, the polymerizable monomer 104 can include a polymerizable monomer represented by Chemical formula 1 or a polymerizable monomer represented by Chemical formula 2,

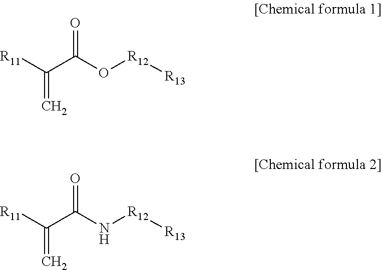

in Chemical formula 1 and Chemical formula 2,
R₁₁ is hydrogen group or C1 to C6 alkyl group,
R₁₂ is a substituted or unsubstituted C1 to C20 alkylene group, and
R₁₃ is —OH or —NH₂.

In one embodiment, the polymerizable monomer represented by Chemical formula 1 is, for example, aminoethyl (meth)acrylate, hydroxyethyl (meth)acrylate, aminopropyl (meth)acrylate or hydroxypropyl (meth)acrylate.

In one embodiment, the polymerizable monomer represented by Chemical formula 2 is, for example, N-(2-aminoethyl)(meth)acrylamide, N-(2-aminoethyl)aminopropyl (meth)acrylamide, N-hydroxyethyl(meth)acrylamide or N-hydroxypropyl(meth)acrylamide.

In this embodiment, the polymerization initiator 102 (having an initiator group for polymerization reaction) fixed on the surface of the substrate 100 can react with the polymerizable monomer 104, to carry out a polymerization reaction, so as to form the branched polymer 108. So far, the step of grafting the branched polymer 108 on the surface of the substrate 100 is completed. In this embodiment, the polymerization reaction is, for example, an atom transfer radical polymerization (ATRP) reaction. That is, the branched polymer 108 of the disclosure is a product obtained through an ATRP reaction. The ATRP reaction can suppress side reactions and premature chain termination reactions that occur in general radical polymerization, and thus the growth of the polymerization can be uniformly performed. Since the branched polymer 108 is formed by the ATRP reaction, the branched polymer of the disclosure can have a structure similar to a carp flag (as shown in FIG. 1), a side chain of a repeating unit of the branched polymer 108 represents each carp of the carp flag, and each side chain has the group 104a capable of binding to the oligonucleotide. Thus, each side chain can bind to the oligonucleotide. In this embodiment, the oligonucleotide is, for example, a linker used for nucleic acid synthesis.

In one embodiment, the branched polymer 108 is, for example, a polymer represented by the following formula (A) or a branched polymer represented by formula (B).

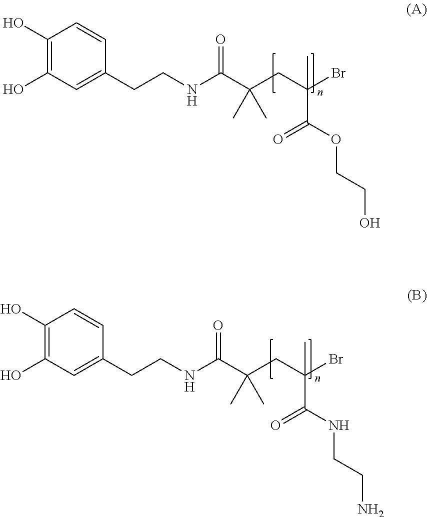

In this embodiment, since the end of the branched polymer can bind to the surface of the substrate, and each side chain of the repeating unit of the branched polymer can bind to the oligonucleotide, it can not only improve the immobilization efficiency of oligonucleotide, but also provide high-density distribution of oligonucleotides on the surface of the substrate.

### Second Embodiment

It must be noted here that the following embodiments follow some content of the foregoing embodiments, and descriptions of the same technical content are omitted. For the description of omitted parts, reference can be made to the foregoing embodiments, and the following embodiments will not be repeated.

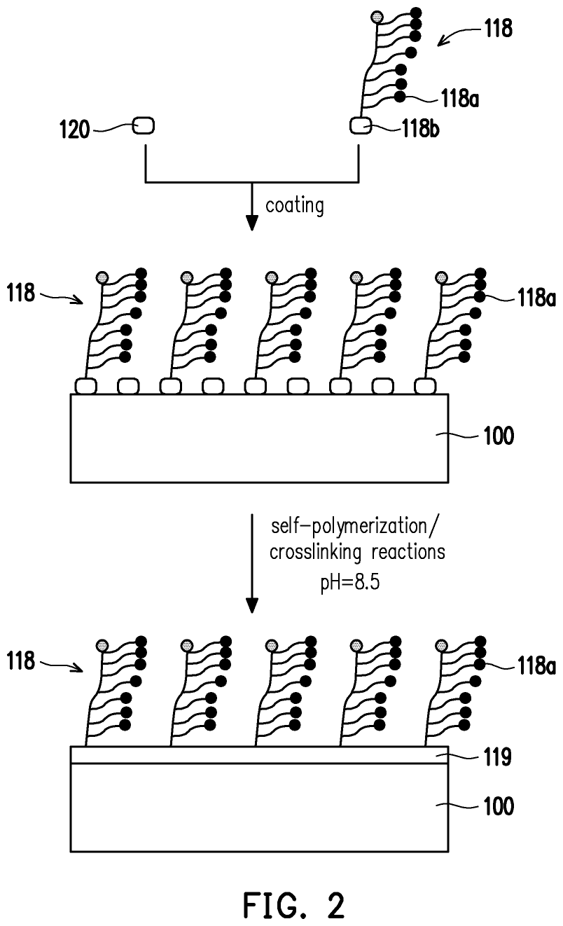

FIG. 2 is a flow chart of modifying a surface of a substrate according to a second embodiment of the disclosure.

First, a substrate 100 is provided. Then, a branched polymer 118 is grafted on a surface of the substrate 100. In one embodiment, the step of grafting the branched polymer 118 on the surface of the substrate 100 includes coating a modification composition including the branched polymer 118 on the surface of the substrate 100. In this embodiment, the side chain of the repeating unit of the branched polymer 118 of the disclosure has a group 118a that can bind to an oligonucleotide, and one end of the branched polymer has a group 118a that can bind to the surface of the substrate.

Specifically, the branched polymer of the disclosure binds to the surface of the substrate 100 through its terminal group. In one embodiment, the group 118b at the end of the branched polymer 118 and capable of binding to the surface of the substrate is, for example, —OH group. In one embodiment, the group 118b at the end of the branched polymer 118 and capable of binding to the surface of the substrate is, for example, a group having a dopamine structure. The group having a dopamine structure can be fixed on the surface of the substrate through a phenolic hydroxyl group.

In this embodiment, the side chain of the repeating unit of the branched polymer 118 of the disclosure has a group 118a capable of binding to the oligonucleotide. In one embodiment, the group 118a of the side chain of the repeating unit which is capable of binding to the oligonucleotide can include —NH₂ group or —OH group.

In one embodiment, the repeating unit of the branched polymer 118 can include a repeating unit derived from a (meth)acrylate-based polymerizable monomer or a (meth)acrylamide-based polymerizable monomer.

In one embodiment, the repeating unit of the branched polymer 118 can include a repeating unit represented by Chemical formula 3 or a repeating unit represented by Chemical formula 4,

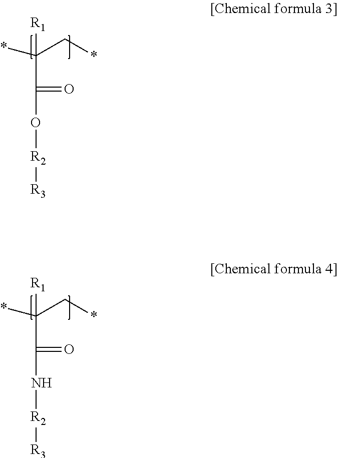

in Chemical formula 3 and Chemical formula 4,
R₁ is hydrogen group or methyl group,
R₂ is substituted or unsubstituted C1 to C20 alkylene group, and
R₃ is —OH or —NH₂.

In this embodiment, the branched polymer 118 of the disclosure is formed, for example, through a polymerization reaction of a polymerization initiator and a polymerizable monomer.

In one embodiment, the polymerization initiator is, for example, a halide-based compound or an acyl halide-based compound. In one embodiment, the polymerization initiator is, for example, a halide-based compound or an acyl halide-based compound having a group 118b at one end thereof and capable of binding to the surface of the substrate. In one embodiment, the polymerization initiator is, for example, 2-bromoisobutyryl catechol derivatives. In one embodiment, the polymerization initiator is, for example, 2-bromoisobutyryl dopamine (DA-BiBB) compound, which has the following structure.

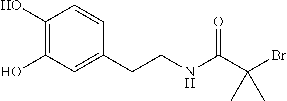 [SMILES: CC(C)(Br)C(=O)NCCc1ccc(O)c(O)c1]

In one embodiment, the polymerizable monomer includes a polymerizable monomer represented by Chemical formula 1 or a polymerizable monomer represented by Chemical formula 2,

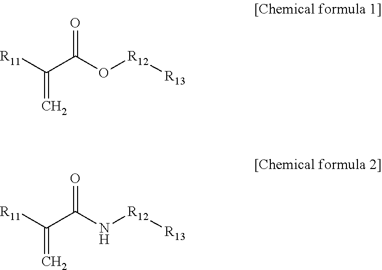

in Chemical formula 1 and Chemical formula 2,
R₁₁ is hydrogen group or C1 to C6 alkyl group,
R₁₂ is a substituted or unsubstituted C1 to C20 alkylene group, and
R₁₃ is —OH or —NH₂.

In one embodiment, the polymerizable monomer represented by Chemical formula 1 is, for example, aminoethyl (meth)acrylate, hydroxyethyl (meth)acrylate, aminopropyl (meth)acrylate or hydroxypropyl (meth)acrylate. In one embodiment, the polymerizable monomer represented by Chemical formula 2 is, for example, N-(2-aminoethyl)(meth)acrylamide, N-(2-aminoethyl)aminopropyl (meth)acrylamide, N-hydroxyethyl(meth)acrylamide or N-hydroxypropyl(meth)acrylamide.

In this embodiment, the branched polymer 118 of the disclosure is formed, for example, through an ATRP reaction of a polymerization initiator and a polymerizable monomer. That is, the branched polymer 118 of the disclosure is a product obtained through an ATRP reaction. The ATRP reaction can suppress side reactions and premature chain termination reactions that occur in general radical polymerization, and thus the growth of the polymerization can be uniformly performed. Since the branched polymer 118 is formed by the ATRP reaction, the branched polymer 118 can have a carp flag structure (as shown in FIG. 2), the side chain of the repeating unit of the branched polymer 118 represents each carp of the carp flag, and each side chain has a group 118a capable of binding to the oligonucleotide. Thus, each side chain can bind to the oligonucleotide. In this embodiment, the oligonucleotide is, for example, a linker used for nucleic acid synthesis.

In one embodiment, the branched polymer 118 is, for example, a polymer represented by the following formula (A) or a polymer represented by formula (B).

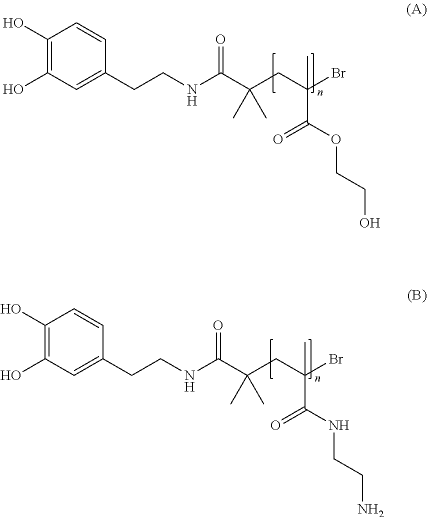

In one embodiment, when the group 118b at the end of the branched polymer 118 and capable of binding to the surface of the substrate is a group having a dopamine structure, the modification composition including the branched polymer 118 of the disclosure can further include additional dopamine 120. In this embodiment, since the additional dopamine 120 does not polymerize with the branched polymer 118 but only binds to the surface of the substrate, the additional dopamine 120 can be used to control the spacing between the branched polymers 118 on the substrate 100, and the spacing further controls the length of subsequently synthesized nucleic acid. In one embodiment, the weight ratio of the additional dopamine 120 in the modification composition to the dopamine in the branched polymer 118 is, for example, 0.1-20. In another embodiment, the weight ratio of the additional dopamine 120 in the modification composition to the dopamine in the branched polymer is, for example, 1-10. Within the aforementioned range, high accuracy of immobilized oligonucleotides can be maintained while effectively controlling the length of subsequently synthesized nucleic acid.

In one embodiment, when the group 118b at the end of the branched polymer 118 and capable of binding to the surface of the substrate is a group having a dopamine structure, after the modification composition is coated on the surface of the substrate 100, the modification composition may further undergo a self-crosslinking reaction in an alkaline environment. Accordingly, a dense coating 119 is formed on the surface of the substrate, thereby improving the binding force of the branched polymer 118 to the substrate 100.

In this embodiment, since the end of the branched polymer can bind to the surface of the substrate, and each side chain of the repeating unit of the branched polymer can bind to the oligonucleotide, it can not only improve the immobilization efficiency of oligonucleotide, but also provide high-density distribution of oligonucleotides on the surface of the substrate.

### Third Embodiment

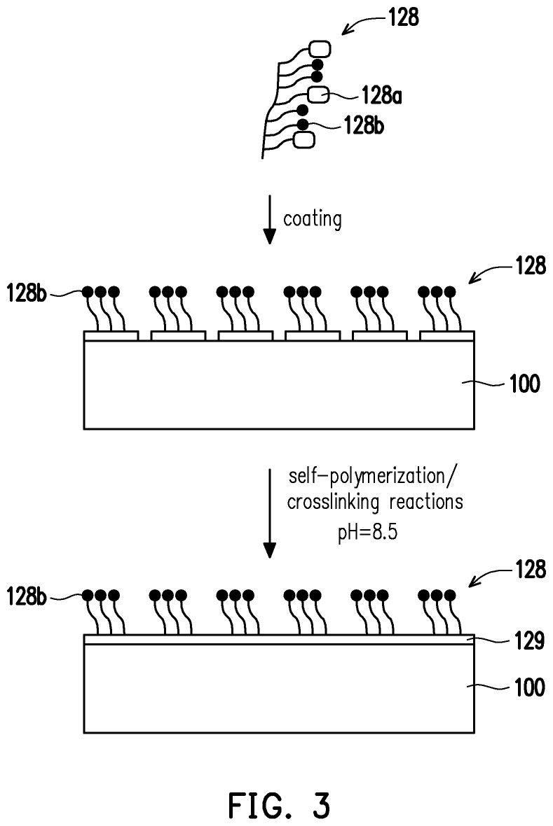

FIG. 3 is a flow chart of modifying a surface of a substrate according to a third embodiment of the disclosure.

First, a substrate 100 is provided. Then, a branched polymer 128 including a first repeating unit and a second repeating unit is grafted on a surface of the substrate 100. Specifically, a modification composition including the aforementioned branched polymer 128 is coated on the surface of the substrate 100. In this embodiment, a side chain of the first repeating unit of the branched polymer 128 of the disclosure has a group 128a capable of binding to the surface of the substrate, and a side chain of the second repeating unit of the branched polymer has a group 128b capable of binding to an oligonucleotide.

Specifically, the branched polymer 128 of the disclosure binds to the surface of the substrate 100 through the group 128a of the side chain of the first repeating unit which can bind to the surface of the substrate. In one embodiment, the group 128a of the side chain of the first repeating unit which can bind to the surface of the substrate can include —OH group. In one embodiment, the group 128a of the side chain of the first repeating unit which can bind to the surface of the substrate is, for example, a group having a catechol structure. The group having a catechol structure can be fixed on the surface of the substrate through a phenolic hydroxyl group.

In one embodiment, the first repeating unit can include a repeating unit derived from a (meth)acrylate-based polymerizable monomer or a repeating unit derived from a (meth)acrylamide-based polymerizable monomer.

In an embodiment, the first repeating unit can include a repeating unit represented by Chemical formula 5 or a repeating unit represented by Chemical formula 6,

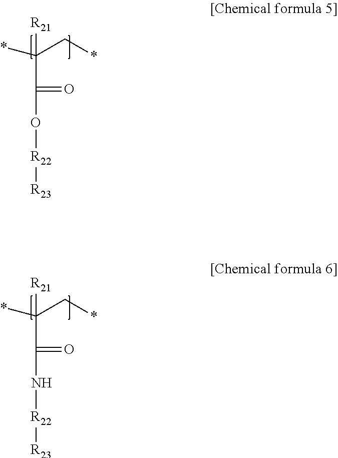

in Chemical formula 5 and Chemical formula 6,
R₂₁ is hydrogen group or methyl group,
R₂₂ is a direct bond or a substituted or unsubstituted C2 to C20 alkylene group, and
R₂₃ is the group capable of binding to the surface of the substrate.

In one embodiment, R₂₃ is a group having a catechol structure.

In this embodiment, the side chain of the second repeating unit of the branched polymer 128 of the disclosure has the group 128b capable of binding to the oligonucleotide. The branched polymer 128 of the disclosure binds to the oligonucleotides by the group 128b of the side chain of the second repeating unit that can bind to the oligonucleotide. In one embodiment, the group 128b of the side chain of the second repeating unit that can bind to the oligonucleotide can include —NH₂ group or —OH group.

In one embodiment, the second repeating unit can include a repeating unit derived from a (meth)acrylate-based polymerizable monomer or a repeating unit derived from a (meth)acrylamide-based polymerizable monomer.

In one embodiment, the second repeating unit includes a polymerizable monomer represented by Chemical formula 7, a polymerizable monomer represented by Chemical formula 8 or a combination thereof.

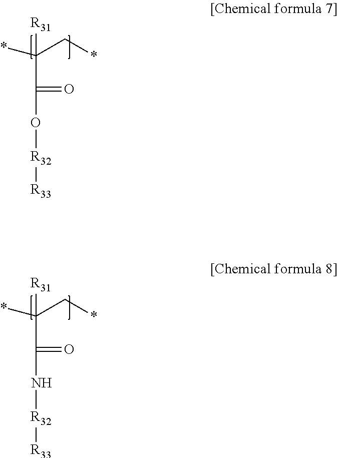

in Chemical formula 7 and Chemical formula 8,
R₃₁ is hydrogen group or methyl group,
R₃₂ is a substituted or unsubstituted C2 to C20 alkylene group, and
R₃₃ is —OH or —NH₂.

In one embodiment, the branched polymer 128 of the disclosure is, for example, a copolymerized branched polymer formed by the polymerizable monomer of the first repeating unit and the polymerizable monomer of the second repeating unit through a radical polymerization. In one embodiment, the polymerizable monomer of the first repeating unit is, for example, methacrylamide dopamine, and the polymerizable monomer of the second repeating unit is, for example, aminoethyl methacrylamide.

In one embodiment, the branched polymer is a branched polymer represented by the following formula (C).

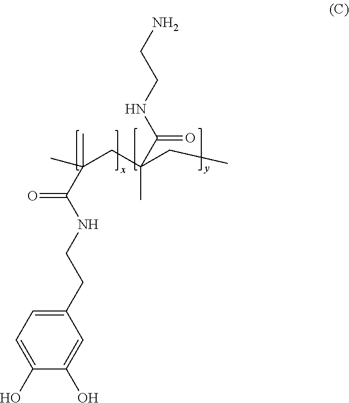

In one embodiment, when the side chain of the group of the first repeating unit of the branched polymer 128 which can bind to the surface of the substrate is a group having a catechol structure, after the modification composition is coated on the surface of the substrate, the modification composition can further undergo a self-crosslinking reaction in an alkaline environment. Accordingly, a dense coating 129 on the surface of the substrate, thereby improving the binding force of the branched polymer and the substrate.

In one embodiment, two polymerizable monomers, methacrylamide dopamine and aminoethyl methacrylamide, can be subjected to a radical polymerization to obtain a copolymerized branched polymer (called PDA-co-PAEMA). Next, after dissolving the branched polymer PDA-co-PAEMA in a suitable solvent to prepare a mixed solution, the mixed solution is coated on a metal surface. Then, a buffer is added to adjust the pH value to about 8.5, and then the dopamine group in the copolymerized branched polymer PDA-co-PAEMA starts self-polymerization and cross-linking reactions. Finally, a dense coating of the branched polymer PDA-co-PAEMA is formed on the metal surface.

In this embodiment, since the side chain of the first repeating unit of the branched polymer 128 can bind to the surface of the substrate, and each side chain of the second repeating unit of the branched polymer can bind to the oligonucleotide. Therefore, it can not only improve the immobilization efficiency of oligonucleotide, but also provide high-density distribution of oligonucleotides on the surface of the substrate.

### [Method for Immobilizing Oligonucleotide of the Disclosure]

The method for immobilizing oligonucleotide of the disclosure includes the following steps.

First, a modified surface of a substrate is provided. In this embodiment, the substrate is a substrate modified by the method of any one of the above-mentioned first embodiment to the third embodiment.

Next, oligonucleotides are brought into to contact with the modified surface of the substrate, wherein the branched polymer on the surface of the substrate binds to the oligonucleotides through the group of each side chain of the repeating unit that can bind to the oligonucleotide, thereby immobilizing the oligonucleotides on the surface of the substrate.

In this embodiment, since the surface of the substrate is modified by the branched polymer, and each side chain of the repeating unit of the branched polymer on the surface of the substrate can bind to the oligonucleotide, the method not only can improve the immobilization efficiency of oligonucleotide and also provide high density distribution of oligonucleotides on the surface of the substrate.

Hereinafter, the disclosure will be described more specifically with reference to examples. Although the following examples are described, the materials used, their amounts and ratios, processing details, processing flow, and the like can be appropriately varied without departing from the scope of the invention. Therefore, the invention should not be limitedly interpreted on the basis of the experiments described hereinafter.

### Experimental Example 1

FIG. 1 is a flow chart of modifying a surface of a metal substrate according to the Experimental example 1.

### [Synthesis of DA-DiBB]

First, dopamine (DA) (400 mg; 2.10 mmol) and 2-bromoisobutyryl bromide (BiBB) (0.13 ml; 1.05 mmol) were dissolved in dimethyl formamide (DMF) (20 ml), and then triethylamine (1.05 mmol) was added and stirred at room temperature under nitrogen atmosphere for 3 hours. After that, DA-BiBB compound (that is, the compound having a dopamine group and using BiBB as ATRP initiator group) was obtained, which has the following structure.

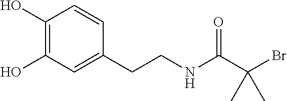 [SMILES: CC(C)(Br)C(=O)NCCc1ccc(O)c(O)c1]

### [Polydopamine Coating of DA-BiBB Compound on Metal Surface by Self-Polymerization and Cross-Linking Reactions]

Next, the above-mentioned DA-BiBB mixture from which the DMF solvent was not removed and tris(hydroxymethyl)aminomethane (Tris) buffer (4.0 mmol) were dissolved in 100 ml of deionized water and mixed. The mixed solution was injected into a 3D printed metal microfluidic channel (or a 3D printed metal sheet was soaked in the mixed solution), so that the mixed solution was coated on the metal surface. Then, the pH value of the mixed solution was adjusted to about 8.5 and the mixed solution was shaken and vibrated at room temperature for 24 hours, to form a coating of polydopamine polymer having ATRP initiator group (i.e., BiBB) on the metal surface of the metal microfluidic channel.

### [Grafting Polyhydroxyethyl Methacrylate (PHEMA) on the Surface of the Metal Microfluidic Channel]

Methanol (10 mL), deionized water (10 mL), ascorbic acid [(+)-sodium L-ascorbate] (354 g), 2,2′-dipyridyl (6 mg) and hydroxyethyl methacrylate (HEMA) monomer (27 mL) were added into a three-necked bottle and mixed to form a mixed solution. Then, the above-mentioned 3D printed metal microfluidic channel (with polydopamine polymer having ATRP initiator group on the surface thereof) was soaked in the mixed solution, and oxygen in the solution was removed through a freeze-thaw cycle. After that, CuBr (4 mg) was added and ATRP reaction was carried out at room temperature under nitrogen for 24 hours, to form polyhydroxyethyl methacrylate (PHEMA) on the metal surface.

### Experimental Example 2

FIG. 2 is a flow chart of modifying a surface of a metal substrate according to the Experimental example 2.

### [Synthesis of DA-DiBB]

First, dopamine (DA) (400 mg; 2.10 mmol) and 2-bromoisobutyryl bromide (BiBB) (0.13 ml; 1.05 mmol) were dissolved in dimethyl formamide (DMF) (20 ml), and then triethylamine (1.05 mmol) was added and stirred at room temperature under nitrogen atmosphere for 3 hours. After that, DA-BiBB compound (that is, the compound having a dopamine group and using BiBB as ATRP initiator group) was obtained.

### [Synthesis of DA-PHEMA Polymer]

The above-mentioned DA-BiBB mixture from which the DMF solvent was not removed was added into a three-necked flask, and then ascorbic acid [(+)-sodium L-ascorbate] (354 g), 2,2′-bipyridine (6 mg), methacrylic acid hydroxyethyl ester (HEMA) monomer (27 mL) were added. Then, oxygen in the mixture was removed through a freeze-thaw cycle. After that, CuBr (4 mg) was added to the mixture and ATRP reaction was carried out at room temperature under nitrogen for 24 hours, to obtain polyhydroxyethyl methacrylate polymer with dopamine end groups (DA-PHEMA).

### [Grafting DA-PHEMA Polymer on the Surface of the Metal Microfluidic Channel]

Dopamine (3.6 g), 100 ml of deionized water and Tris buffer (480 mg) were additionally added to the above solution containing DA-PHEMA polymer and mixed. Next, the 3D printed metal microfluidic channel was placed in the mixed solution. The mixed solution was kept stirring (not just standing) and subjected to a surface treatment at room temperature for 24 hours, to form a coating with dopamine and DA-PHEMA polymer on the surface of the metal microfluidic channel. So far, the grafting on the surface of the metal microchannel has been completed.

Since the branched polymer of the disclosure can bind to the surface of the substrate, and each side chain of the repeating unit of the branched polymer can bind to the oligonucleotide, it can not only improve the immobilization efficiency of oligonucleotide, but also provide high density distribution of oligonucleotides on the surface of the substrate. Therefore, the method for immobilizing oligonucleotide of the disclosure is suitable for nucleic acid synthesis.

It will be apparent to those skilled in the art that various modifications and variations can be made to the structure of the disclosed embodiments without departing from the scope or spirit of the disclosure. In view of the foregoing, it is intended that the disclosure cover modifications and variations of this disclosure provided they fall within the scope of the following claims and their equivalents.

What is claimed is:

1. A method for modifying a surface of a substrate, comprising:
providing a substrate; and
grafting a branched polymer on a surface of the substrate, wherein a side chain of a repeating unit of the branched polymer comprises a group capable of binding to an oligonucleotide, and one end of the branched polymer comprises a group capable of binding to the surface of the substrate.

2. The method of claim 1, wherein the group capable of binding to an oligonucleotide comprises —NH₂ group or —OH group.

3. The method of claim 1, wherein the group capable of binding to the surface of the substrate comprises a group having a dopamine structure.

4. The method of claim 3, wherein after grafting the branched polymer on the surface of the substrate, the branched polymer further undergoes a self-crosslinking reaction in an alkaline environment.

5. The method of claim 1, wherein the repeating unit comprises a repeating unit derived from a polymerizable monomer of (meth)acrylate or a repeating unit derived from a polymerizable monomer of (meth)acrylamide.

6. The method of claim 1, wherein grafting the branched polymer on the surface of the substrate comprises:
coating a first modification composition comprising a polymerization initiator on the surface of the substrate, wherein one end of the polymerization initiator has the group capable of binding to the surface of the substrate; and
adding a second modification composition comprising a polymerizable monomer to the first modification composition on the surface of the substrate, to carry out a polymerization reaction, so as to form the branched polymer,
wherein the polymerizable monomer has the group capable of binding to an oligonucleotide.

7. The method of claim 6, wherein the polymerizable monomer comprises a polymerizable monomer represented by Chemical formula 1 or a polymerizable monomer represented by Chemical formula 2, 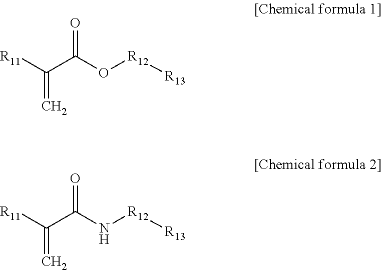
in Chemical formula 1 and Chemical formula 2,
R₁₁ is hydrogen group or C1 to C6 alkyl group,
R₁₂ is a substituted or unsubstituted C1 to C20 alkylene group, and
R₁₃ is —OH or —NH₂.

8. The method of claim 6, wherein the group of the polymerization initiator capable of binding to the surface of the substrate comprises a group having a dopamine structure.

9. The method of claim 8, wherein after coating the first modification composition on the surface of the substrate, the first modification composition further undergoes a self-crosslinking reaction in an alkaline environment.

10. The method of claim 1, wherein grafting the branched polymer on the surface of the substrate comprises:
coating a modification composition comprising the branched polymer on the surface of the substrate.

11. The method of claim 10, wherein the modification composition further comprises additional dopamine.

12. The method of claim 11, wherein the group capable of binding to the surface of the substrate comprises a group having a dopamine structure, and
a weight ratio of the additional dopamine in the modification composition to dopamine in the branched polymer is 0.1-20.

13. The method of claim 5, wherein the repeating unit of the branched polymer comprises a repeating unit represented by Chemical formula 3 or a repeating unit represented by Chemical formula 4, 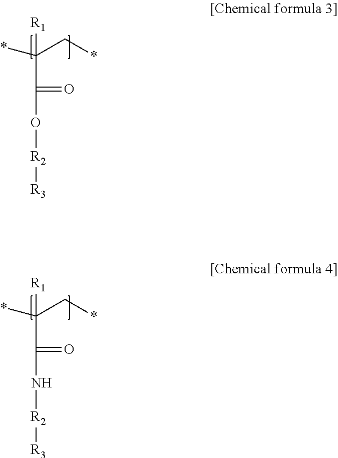
in Chemical formula 3 and Chemical formula 4,
R₁ is hydrogen group or methyl group,
R₂ is substituted or unsubstituted C1 to C20 alkylene group, and
R₃ is —OH or —NH₂.

14. A method for modifying a surface of a substrate, comprising:
providing a substrate; and
grafting a branched polymer comprising a first repeating unit and a second repeating unit on a surface of the substrate, wherein a side chain of the first repeating unit of the branched polymer has a group capable of binding to the surface of the substrate, and a side chain of the second repeating unit of the branched polymer has a group capable of binding to an oligonucleotide.

15. The method of claim 14, wherein the group capable of binding to an oligonucleotide comprises —NH₂ group or —OH group.

16. The method of claim 14, wherein each of the first repeating unit and the second repeating unit independently comprises a repeating unit derived from a polymerizable monomer of (meth)acrylate or a repeating unit derived from a polymerizable monomer of (meth)acrylamide.

17. The method of claim 16, wherein the first repeating unit comprises a repeating unit represented by Chemical formula 5 or a repeating unit represented by Chemical formula 6, and the second repeating unit comprises a repeating unit represented by Chemical formula 7 or a repeating unit represented by Chemical formula 8, 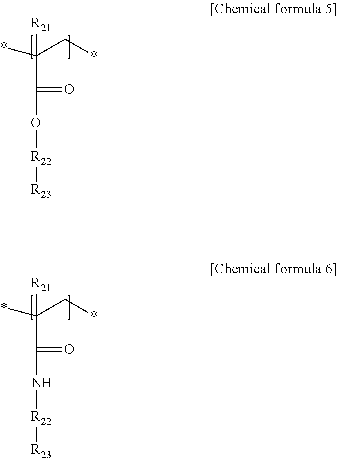
in Chemical formula 5 and Chemical formula 6,
R₂₁ is hydrogen group or methyl group,
R₂₂ is a direct bond or a substituted or unsubstituted C2 to C20 alkylene group, and
R₂₃ is the group capable of binding to the surface of the substrate, 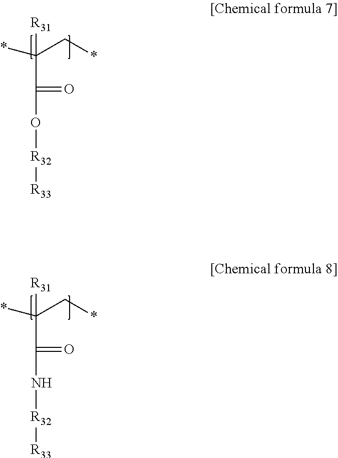
in Chemical formula 7 and Chemical formula 8,
R₃₁ is hydrogen group or methyl group,
R₃₂ is a substituted or unsubstituted C2 to C20 alkylene group, and
R₃₃ is —OH or —NH₂.

18. The method of claim 17, wherein the group capable of binding to the surface of the substrate comprises a group having a catechol structure.

19. The method of claim 18, wherein after grafting the branched polymer on the surface of the substrate, the branched polymer further undergoes a self-crosslinking reaction in an alkaline environment.

20. A method for immobilizing oligonucleotide, comprising:
providing a substrate modified by using the method of claim 1; and
contacting the surface of the substrate with oligonucleotides.
